(19) United States
(12) Patent Application Publication
(10) Pub. No.: US 2016/0114116 A1
(43) Pub. Date: Apr. 28, 2016

(54) Low-Profile Bifurcated Bilateral Endotracheal-Endobronchial Tube and Methods of Using

(71) Applicant: Wake Forest University Health Sciences, Winston-Salem, NC (US)

(72) Inventor: Adele K. Evans, Winston-Salem, NC (US)

(21) Appl. No.: 14/923,688

(22) Filed: Oct. 27, 2015

Related U.S. Application Data
(60) Provisional application No. 62/068,924, filed on Oct. 27, 2014.

(51) Int. Cl. A61M 16/04

(52) U.S. Cl. CPC A61M 16/0486; A61M 16/0475; A61M 16/0434; A61M 16/0404; A61M 2205/3334

(57) ABSTRACT

Low-profile bifurcated bilateral endotracheal-endobronchial tube devices and methods for using the devices are provided for airway management in patients with special anatomical or medical issues or for certain complex procedures. Specific endobronchial ventilation, as opposed to endotracheal ventilation, can better facilitate a tracheal repair by protecting the trachea and simultaneously providing an airtight conduit to the bronchi, for example.

### RELATED APPLICATIONS

The present application claims priority to U.S. Provisional Patent Application No. 62/068,924, filed Oct. 27, 2014. The disclosure of U.S. Provisional Patent Application No. 62/068,924 is hereby incorporated by reference in its entirety herein.

### FIELD

The present invention relates to the medical field. Disclosed exemplary embodiments include devices for a low-profile bifurcated bilateral endotracheal-endobronchial tube and methods of using such a device.

### BACKGROUND

Some medical procedures may be better performed with the ability to expose or gain access to the carina or other respiratory structures without compromising delivery of gases to functional lung tissues. A variety of considerations may influence the choice of devices for management of a particular patient airway. In some cases, devices with special configurations may be required. Construction of a custom bifurcated endobronchial tube has previously been attempted using high-profile tubes assembled with suture materials. Still, a low-profile, atraumatic device is unavailable, and is needed to allow for providing an endotracheal-endobronchial tube suitable for a large variety of procedures requiring such devices. For example, in various medical circumstances a low-profile bifurcated bilateral endotracheal-endobronchial (ET-EB) tube device is needed for airway management before, during, or after surgical or other medical procedures, and in some cases even for long-term maintenance of the airway and associated anatomical structures.

### SUMMARY

In some aspects, the present invention provides a low-profile device for managing a patient airway, including a single-lumen tracheal tube that bifurcates to two single-lumen bronchial tubes to function as an endotracheal-endobronchial (ET-EB) tube device. In an embodiment, the ET-EB tube of the invention has substantially smooth exterior surfaces providing a low-profile exterior. In some embodiments the ET-EB tube device further includes substantially smooth interior surfaces.

In some embodiments, a low-profile ET-EB tube of the invention is designed for the anatomy of a particular patient. In some embodiments, a low-profile ET-EB tube of the invention further includes an opening to accommodate the level of the right superior lobar bronchus as it branches from the right main bronchus. In some embodiments, the opening is on the bifurcated part of the device.

In some embodiments, a low-profile ET-EB tube of the invention further includes an external cuff or cuffs positioned at the distal end of the bronchial tubes, for example. A cuff may also be positioned at various points along the tracheal tube portion of the device. In some embodiments an ET-EB tube of the invention may further include an internal valve, positioned in one or both of the bronchial tubes, for example. In some embodiments, an internal valve may be associated with an internal cuff.

In some embodiments of a low-profile ET-EB tube of the invention, the internal diameter of the tracheal tube part of the device is between 2.0-8.5 mm. The size and/or ratio between tracheal and bronchial diameters may be designed to fit within neonatal, early childhood, pediatric, adolescent, adult, or large or enlarged adult size trachea and bronchi as is needed.

In some aspects, a low-profile ET-EB tube device of the invention is substantially airtight at the junction where the tracheal tube bifurcates to two bronchial tubes. In some embodiments, the bronchial tubes of the ET-EB tube of the invention are asymmetrical. In some embodiments, the ET-EB tube device of the invention is substantially transparent.

In some aspects, the invention provides a method for managing an airway, comprising placement of a device having a single-lumen tube that bifurcates to two single-lumen tubes to function as an endotracheal-endobronchial (ET-EB) device. In an embodiment the ET-EB device has substantially smooth exterior surfaces providing a low-profile exterior. Some methods may further include selecting or customizing the low-profile ET-EB tube device to comprise an opening corresponding to a superior lobar bronchus of the patient. Some methods further include using an inflatable cuff positioned to secure the device in position once it is placed. Some methods further include the use of an internal valve, positioned in one or both bronchial tubes of the ET-EB tube device, to limit gas flow in a particular segment of the device or an external valve to secure the device in a particular portion of the patient's airway.

In certain embodiments of the method the low-profile ET-EB device is used to manage the airway of a patient afflicted with a congenital defect, traumatic injury, surgical, cancerous, infectious, or other condition that compromises the proper functioning of the trachea and/or bronchi and/or to manage the airway of a patient before, during, or after a cardio-thoracic procedure. In some embodiments, gases are delivered selectively to one lung. In other embodiments, the method may further include recruitment and/or recovery of a lung during or following the use of ECMO or cardiopulmonary bypass. Alternatively, methods for airway management may be employed to facilitate avoidance of ECMO or cardiopulmonary bypass.

Various non-limiting embodiments of the devices and methods of the invention are described below.

### BRIEF DESCRIPTION OF THE FIGURES

FIG. 1 shows the bronchial tubes and distal end of the tracheal tube of an endotracheal-endobronchial tube device according to an embodiment of the invention.

FIG. 2 is an illustration of a typical adult human trachea and bronchi.

FIG. 3 shows several views of the bronchial tubes and distal end of the tracheal tube of an endotracheal-endobronchial (ET-EB) tube device according to one embodiment of the invention.

FIG. 4 shows an embodiment of an ET-EB tube, as constructed for a neonatal patient described in Example 1.

FIG. 5 shows a side-by-side comparison of the ET-EB tube of FIG. 4 (lower) and a lower-profile endotracheal-endobronchial tube (upper).

FIG. 6 shows an embodiment of the ET-EB tube device of Example 1 as illustrated in FIG. 4.

FIG. 7 shows a closeup of the distal end of the ET-EB device shown in FIG. 6.

FIG. 8 shows one embodiment of a low-profile ET-EB tube.

FIG. 9 shows a barium esophagogram of the patient described in Example 1.

FIG. 10 shows a chest X-ray of the patient described in Example 1, with an ET-EB tube in place.

FIG. 11 shows CT images from a neonatal airway with tracheo-esophageal fistula that were used to determine appropriate dimensions for modeling an airway via 3D printing.

FIG. 12 shows a 3D-printed model neonatal airway with a tracheo-esophageal fistula, for use in a model for placement testing of a prototype ET-EB tube of the invention.

FIGS. 13A and 13B show testing results for placement of a prototype ET-EB tube of the invention, as compared to a standard endotracheal tube (ETT) (i.e., a tube that does not include bronchial tubes). Placement time is shown in FIG. 13A and subjective ease of placement is shown in FIG. 13B.

FIG. 14 shows the static pressure relationships of flow and resistance for a prototype ET-EB tube of the invention, as compared to a standard endotracheal tube (ETT).

### DETAILED DESCRIPTION

The methods, systems, and devices of the present invention may be advantageously utilized in medical treatment protocols for humans and/or in veterinary applications.

The following description recites various aspects and embodiments of the present invention. No particular embodiment is intended to define the scope of the invention. Rather, the embodiments merely provide non-limiting examples of various methods, devices, and systems that are at least included within the scope of the invention. The description is to be read from the perspective of one of ordinary skill in the art; therefore, information well-known to the skilled artisan is not necessarily included.

### DEFINITIONS

As used in this specification, the singular forms “a,” “an,” and “the” include plural referents unless expressly and unequivocally limited to one referent.

“Low-profile,” as used herein, refers to a device having substantially smooth or continuous exterior surfaces, especially at any point to be positioned or placed distal to the oropharynx, such that potential for resistance and irritation are minimized for passage of the device through the larynx, trachea, and bronchi, despite the presence of any variety of anatomical anomalies. A low-profile ET-EB device, as used herein, is generally characterized by gradual transitions between narrower and broader segments, rather than abrupt changes in diameter. A low-profile ET-EB device may also include distal parts having a broadest outer diameter such that it is narrow enough to pass through the cricoid cartilage of a patient. Optionally, a low-profile ET-EB device also has substantially smooth and/or continuous interior surfaces, such that resistance is likewise minimized for passage of instruments, air, or other substances through the lumen or lumens of the device.

“Substantially smooth surfaces,” or “continuous surfaces,” as used herein, refers to surfaces that may include curves, expansions, or narrowing transitions, but wherein any such transitions are gradual such that they do not include abrupt changes in contour or jagged or irregular features that could introduce unnecessary resistance during placement or adjustment of medical devices. Optionally, a low-profile ET-EB device also has substantially smooth and/or continuous interior surfaces, such that resistance is likewise minimized for passage of instruments, air, or other substances through the lumen or lumens of the device. An external or internal cuff may optionally be included in ET-EB devices of the present invention without significantly disrupting the substantial smoothness of a surface, as the cuffs are soft and maintain a low profile when deflated. The ET-EB tubes of the invention may or may not have an external or internal cuff.

A “cuff,” as used herein, refers to an external or internal inflatable region along parts of an ET-EB tube (e.g., at the distal end of the bronchial tubes or at the proximal end of the tracheal tube). When inflated, a cuff forms a seal against the tracheal wall or bronchial wall (or if internal, against the wall of the tracheal or bronchial portion of the tube). This seal prevents gases from leaking past the cuff and in some circumstances an external cuff facilitates positive pressure ventilation. The seal may also prevent matter such as regurgitated gastric contents from entering the trachea. The ET-EB tubes of the invention may or may not have an external or internal cuff.

A “valve,” as used herein, refers to an internal mechanism within a tube for restricting or allowing air flow. The valve may be an internal cuff, or it may be another type of valve. Specialized types of valves are known. A valve may be a one-way valve, i.e., a valve that may restrict flow to only one direction, or it may completely restrict flow.

A “Murphy eye,” as used herein refers to a common opening in the distal side or wall of an endotracheal tube which allows airflow to continue in the event of the tube opening lying against the tracheal wall or being obstructed in other ways.

Unless otherwise specified, all tube diameters (e.g., sizes) herein refer to the internal diameter of a tube, in millimeters (mm).

### Devices

Airway management before, during, and after surgical and other medical procedures often presents a variety of challenges to medical personnel. Anatomy may vary considerably, even in patients of the same age or size, and a patient's medical condition may further complicate management of the airway. Planned or potential procedures to be performed while the airway is being managed (e.g., surgical or exploratory procedures) may also factor into airway management-related decisions. In some cases, a patient airway must be managed long-term, which presents additional challenges and complications.

In some aspects, the invention described herein provides low-profile endotracheal-endobronchial tube devices and methods for using such devices, to facilitate management of a difficult or complicated patient airway. In some embodiments, the devices and methods of the invention facilitate airway management to optimally accommodate a particular procedure or recovery of the airways from a medical procedure, as in the examples described herein.

In some embodiments, a low-profile bifurcated bilateral endotracheal-endobronchial (ET-EB) tube is a device including a single-lumen tube that bifurcates to two single-lumen tubes. This configuration corresponds to the trachea as it bifurcates distally at the carina to the two bronchi leading to the lungs.

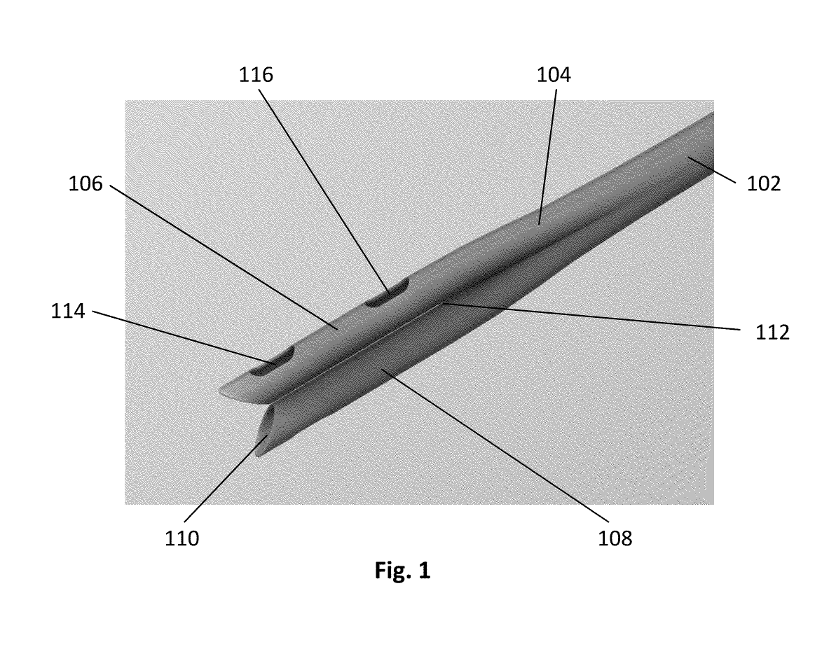

A low-profile bifurcated ET-EB tube of the invention may have substantially smooth or continuous exterior surfaces. A smooth exterior may include transitions, curves, or expansions, but any such features may also incorporate a gradual change in the exterior profile, such that the exterior profile generally does not include the trailing ends of sutures, ridges, juts, or any other jagged or sudden changes in contour or hard edges or irregularities that may provide additional resistance during placement of the device and which may further irritate the respiratory epithelium or other airway tissues during the time that such a device remains in place in a patient airway. Accordingly, a smooth exterior or “low-profile” design may minimize resistance for passage of the device through the larynx, trachea, and into the bronchi. A low-profile device may also minimize irritation of the respiratory epithelium and other airway tissues while the device remains in the airway. For example, standard endotracheal tubes can be difficult to pass through the glottis in some patients. Any added irregularities along the exterior surface of a device tend to exacerbate this problem. By contrast, substantially smooth exterior surfaces on a device can help to alleviate or minimize the problem. As such, a low-profile device is also atraumatic with respect to the anatomical structures it encounters. FIG. 1 illustrates one embodiment of a low-profile ET-EB tube of the invention.

An ET-EB tube may also have substantially smooth interior surfaces. Such smooth interior surfaces may also include transitions, curves, or expansions, but avoid sudden changes in contour or sharp irregularities that may cause medical instruments, air, or other substances that may pass through the ET-EB tube to encounter undesirable and unnecessary resistance. Some embodiments of an ET-EB tube may further include one or more Murphy eyes at the tip of the bronchial parts of the tube.

Furthermore, unlike previously attempted constructions, embodiments of the present invention are airtight at the junction where the tracheal tube portion bifurcates into the bronchial tubes. The lower profile and airtight construction provide improved specific endobronchial ventilation as compared to previous devices.

Embodiments of an ET-EB tube may be any of a wide range of sizes. For example, a pediatric-sized device may have an internal diameter of 4.0 mm in the single-lumen (tracheal) part and a diameter of 3.0 mm in each of the bifurcated (bronchial) parts. By contrast, an adult-sized ET-EB tube may, for example, have an internal diameter of 6-10 mm in the tracheal tube part and a diameter of 4-6 mm in each bronchial tube part. In some embodiments, the internal diameter of the tracheal part of an ET-EB tube device is 2.0, 2.5, 3.0, 3.5, 4.0, 4.5, 5.0, 5.5, 6.0, 6.5, 7.0, 7.5, 8.0, or 8.5 mm. In considering the appropriate size for a particular patient, parts of the device to be placed distal to the cricoid cartilage usually should be narrow enough to pass through that structure. In many pediatric patients, distal parts of an ET-EB tube may need to be even narrower than the cricoid ring to accommodate the narrower subglottic airway. Thus the ratio between tracheal diameter and bronchial diameter of an ET-EB tube is not fixed, but may vary based on the size and condition of the patient in need of airway management. Various combinations of tracheal diameter and bronchial diameter may also be appropriate for particular medical circumstances. For several examples, subglottic edema, mass lesions in the trachea, or aspiration of a foreign body may be important factors in selecting an appropriate sized ET-EB tube for a particular patient.

In some embodiments, an ET-EB tube is of uniform or nearly uniform diameter throughout the single-lumen part (i.e., the tracheal tube part). The bifurcated parts (i.e., bronchial tubes) may also be of uniform or nearly uniform diameter. In other embodiments, the various parts of an ET-EB tube may gradually narrow to a smaller diameter from proximal to distal, or vice versa, or some combination thereof. For example, the tracheal part of an ET-EB tube may broaden from proximal to distal, and the bifurcated parts may narrow from proximal to distal, or vice versa, or both parts may narrow in the same direction, or vice versa, as needed for an individual patient's anatomy and medical condition.

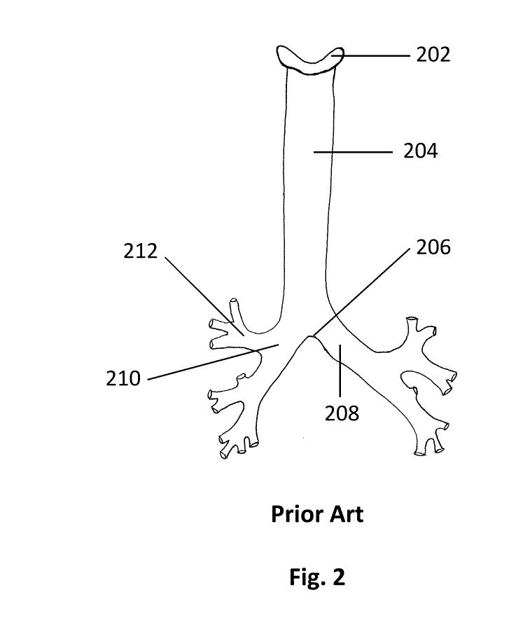

In some embodiments of an ET-EB tube, the bifurcated part of the device (i.e., the bronchial tubes) may be asymmetrical, such that the tube destined for the right main bronchus is shorter than the tube destined for the left main bronchus (or vice versa). This configuration may accommodate the asymmetrical anatomical structure of the lungs. That is, the opening from the right bronchus into the right superior lobar bronchus (as demonstrated in FIG. 2) is proximal to the opening for any other lobar bronchus. A shorter right bronchial tube on the ET-EB tube device may thus allow better delivery of air to all lobes of the right lung. Alternatively, in an ET-EB tube device having a bifurcated part with tubes of equal length, a Murphy-eye may accommodate the superior lobar bronchus, or additional openings may be included in the tube destined for the right bronchus, as shown in FIG. 1 (e.g., if the end of the bifurcated part of such a device is to be placed substantially distal to the opening for the right superior lobar bronchus). An additional, more proximal opening may also be larger (or smaller) than a Murphy eye. In some embodiments, a Murphy-eye may be included in both bronchial branches of an ET-EB tube.

In some embodiments, an ET-EB tube is constructed from materials already known and used in existing endotracheal devices. For example, polypropylene or polyethylene as used in existing approved endotracheal tubes may also be used in the manufacture or construction of an ET-EB tube. A specific example is the MALLINCKRODT® Hi-Lo Oral/Nasal Tracheal Tube Cuffed (Product 86442, Covidien).

Some embodiments of an ET-EB tube may further include an external cuff or an internal valve or both. An external cuff may function to form a seal against the tracheal or bronchial wall when inflated. A cuff may also prevent matter such as regurgitated gastric contents from entering the trachea. Thus a cuff may be positioned on the proximal or distal part of the tracheal tube, for example, or cuffs may be positioned on the bronchial tube parts of the ET-EB tube device. Ideally, a cuff adds minimally to the external profile of the device, as it typically may be constructed from a very soft collapsible material.

Also optionally, an internal valve may be included in some embodiments, as a mechanism or tool for controlling the passage of air in a particular segment of an ET-EB tube. In some medical circumstances, it may be desirable to selectively choose to pass or block air passage, for example at various stages of a procedure. Any mechanism that achieves this objective may be incorporated in an ET-EB tube. For example, a valve may be positioned in the bronchial tube portions of an ET-EB tube so as to allow occlusion of one lung. An internal cuff may serve as a valve, or other types of valve, or anything that can be used to achieve selective occlusion of a particular segment of the ET-EB tube device may be built into the ET-EB tube device or added in modification.

### Methods

In some aspects, methods of using an ET-EB tube device of the invention include placing the device in the trachea and bronchi of a patient in need of airway management. Methods for using an ET-EB tube according to the invention may use many of the same instruments used to assist medical personnel with placement of existing endotracheal tubes or other intubation procedures. For example, a HOPKINS® rod-lens telescope or flexible fiber optic bronchoscope, Storz AIDA® system, jet ventilation, pressure control ventilation, stylet, laryngoscope blade (e.g., Miller, Mac, Benjamin, Parsons, or Wisconsin blade), or other devices (e.g., GLIDESCOPE®) may be used to aid placement of an ET-EB tube of the invention. Accordingly, additional new devices are generally not necessary for adaptation to methods utilizing an ET-EB tube.

In a method of placing an ET-EB tube device, the bifurcation point of the device may be placed such that it rests at or above the carina (i.e., where the trachea bifurcates to the main bronchi) in the patient. Existing intubation instruments, such as stylets and a telescope, may assist medical personnel in guiding the placement of an ET-EB tube bifurcation correctly, i.e., at or above the carina. Optimal selection of devices to aid in placement of an ET-EB tube may depend on the size and condition of the patient. If the ET-EB tube device includes one or more external cuffs, the cuffs may be inflated once the tube is properly placed, such that the tube does not shift out of position (or to minimize shifting), and such that the passage of air or other substances around the periphery of the tube is minimized or eliminated. Again, this may depend on the anatomy and medical condition of the particular patient.

Skilled medical personnel will readily appreciate that air pressures may also need to be adjusted and optimized, both generally and in the case of occlusion of some part of the ET-EB tube device this may be done using an internal valve or valves, to accommodate differences in fluid mechanics as a result of the differences between ventilation with a valve open or closed. The appropriate air pressure adjustments may also depend on the anatomy and medical condition of a particular patient.

In some methods of the invention, an ET-EB tube of the invention may be placed in the trachea and bronchi of a patient in preparation for a cardio-thoracic procedure (e.g., intubation for a surgery, such as open-heart surgery). In some embodiments, an ET-EB tube as optimized for a particular patient may include one or more valves, such that air passage through a particular part of the device may be occluded as needed during stages of a procedure (e.g., one lung isolation). For example, the delivery of air to one lung may be temporarily discontinued by closure of a valve within an ET-EB tube device (e.g., in one branch of the bifurcated part), in order to facilitate better access to specific structures, or to reduce air pressure on some structures, in cardio-thoracic or other thoracic procedures. As a more specific example, a patient may be placed on extracorporeal membrane oxygenation (ECMO) under various circumstances (e.g., pre-operative or post-operative), so as to allow access to one lung for a surgical procedure or to allow a lung to recover from such a procedure. In the latter case, for example, once the lung has had adequate time to recover, the patient may be weaned from ECMO and the lung may be recruited back to a functional state. Using an ET-EB tube may facilitate such a transition, for example by allowing delivery of air to the other lung to continue while limiting delivery of air to the recovering lung.

In some methods of the invention, utilizing an ET-EB tube may be useful when one lung needs to be isolated in order to protect the other lung. For example, if one lung has developed an infection, but the other lung is clear of infection, it is generally desirable to prevent transmission of the infectious agent to the clear lung. Likewise if one lung is bleeding, it is generally desirable to prevent blood from entering the trachea or the other lung. In such cases, an ET-EB tube with specialized valves in the bifurcated tubes may be appropriate.

In other embodiments of methods of the invention, an ET-EB tube device may protect aspects of the airway, such as the trachea, from high gas pressures or from instruments to be passed through the ET-EB tube. For example, an ET-EB tube may facilitate protection of the trachea (i.e., reduce air pressure and provide a protective substitute conduit) while delivery of gases to both lungs is continued. For example, in the case of a laryngotracheal suture line (i.e., tracheo-esophageal fistula repair, laryngotracheo-esophageal cleft, or tracheal resection), high tracheal pressures can be avoided by direct mainstem bronchial ventilation.

Example 1 described herein is a detailed account of a neonatal LTEC repair that successfully utilized three different versions of an ET-EB tube, adapted for the changing patient conditions and growth over a 2 month interval, yielding excellent airway control with pulmonary hygiene, stability of oxygen saturations, and stability of ventilation control throughout the patient's complex course of care, which included multiple surgical procedures.

The skilled medical professional will readily appreciate the wide range of additional medical circumstances in which surgical procedures and other medical methods may benefit from use of an ET-EB tube as described herein. As will be appreciated, the foregoing figures and examples are illustrative, and non-limiting, of the many possible embodiments of the present invention.

One embodiment of a low-profile endotracheal-endobronchial (ET-EB) tube device is illustrated in FIG. 1. In this embodiment, the tracheal part of the tube 102 transitions smoothly as it bifurcates 104 to the right 106 and left 108 bronchial tubes. In this embodiment, the bronchial tubes have beveled tips 110 (i.e., longer at the periphery than in the middle) such that they encourage the bifurcation of the device to split properly to the left and right bronchi when they encounter the carina in a patient. The meeting of the right 106 and left 108 bronchial tubes 112 may be placed to allow positioning of the ET-EB tube device at or just above the carina. This embodiment also demonstrates the optional features of a Murphy eye 114 and an additional, more proximal opening on the right bronchial tube 116 near or just distal to the bifurcation. The additional opening 116 may be larger or smaller than a typical Murphy eye.

A typical illustration of an adult trachea and bronchi are shown in FIG. 2. The cricoid cartilage 202 at the superior aspect of the trachea 204 restricts to some extent the size of instruments entering the trachea, and more so in younger patients. The carina 206 marks the bifurcation of the trachea 204 into the left 208 and right 210 main (or primary) bronchi. As described herein, the carina 206 is where the ET-EB tube bifurcation may be placed (i.e., either at or just above the carina 206). The bronchial tree is typically asymmetrical, such that the right primary bronchus 210 begins to branch further at a level more proximal to the carina 206 than the left primary bronchus 208. The first branch from the right primary bronchus is the right superior lobar bronchus 212. This asymmetry is slightly variable from one patient to another and may be a consideration for selection or construction of an ET-EB tube that is appropriate for a particular patient. Imaging techniques may be used to determine the bronchial anatomy of a particular patient.

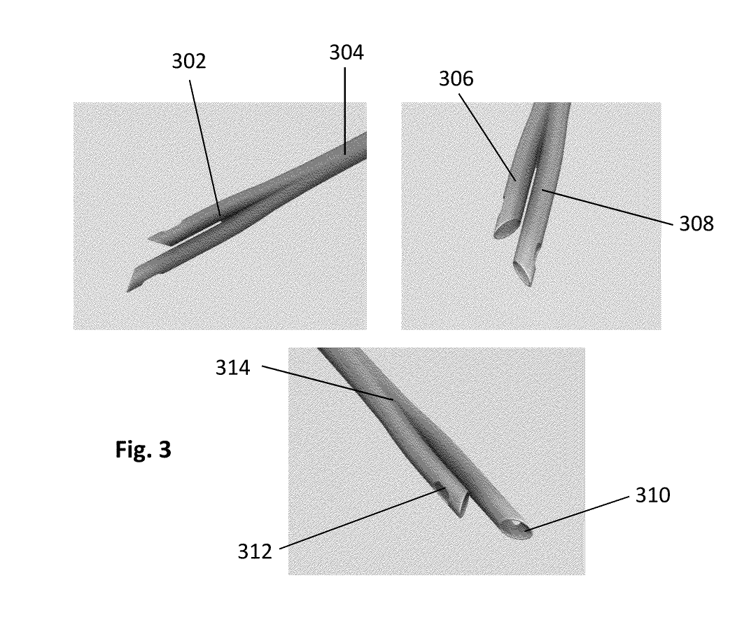

Several views of the distal end of one embodiment of a low-profile ET-EB tube device are shown in FIG. 3. This embodiment bifurcates 302 smoothly from the tracheal tube part 304 into a shorter right bronchial tube 306 and a longer left bronchial tube 308. Again, this configuration corresponds to the asymmetrical bronchial anatomy. Beveled tips 310 encourage proper placement of the right 306 and left 308 bronchial tubes into the right and left bronchi. The low-profile of the device includes a smooth gradual transition 314 from the tracheal part of the device to the bifurcated bronchial parts of the device. The Murphy-eye 312 allows for ventilation of second order bronchial branches off the mainstem bronchi.

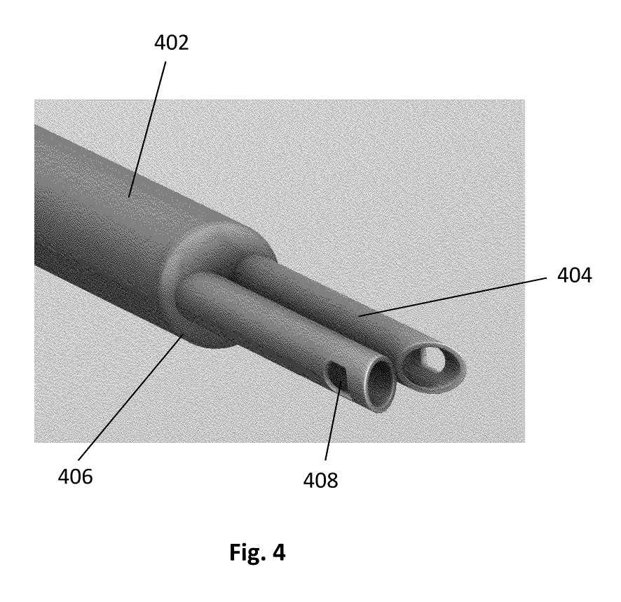

An early embodiment of an ET-EB tube is illustrated in FIG. 4, as constructed for a neonatal patient described in Example 1. The tracheal part of this device 402 was derived from a size 6.0 MALLINCKRODT® endotracheal tube, and the bronchial parts of the device 404 were derived from size 2.5 MALLINCKRODT® endotracheal tubes. The junction of the tracheal part with the bronchial parts 406 was not as smooth or gradual as the low-profile ET-EB tube devices constructed later for the same patient. The bronchial tips of the tubes each included a Murphy-eye 408 as the tips of the original endotracheal tubes.

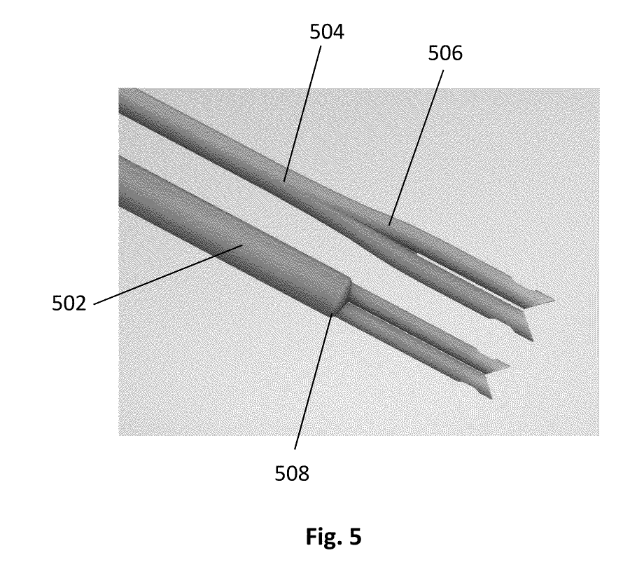

A side-by-side comparison of the first ET-EB tube of Example 1 (illustrated in FIG. 4) and a lower-profile “tooled” ET-EB tube of the invention is shown in FIG. 5. The tracheal part of the first device 502 had a larger diameter with respect to the diameter of the bronchial parts of that device (i.e., tracheal to bronchial ratio) as compared to the low-profile tooled device 504. Also the low-profile tooled device 504 demonstrates a smoother, more gradual transition 506 between the tracheal part and the bifurcation to the bronchial parts than the first device transition 508, providing for ease of placement in a patient's airway and easier passage of instruments through the ET-EB tube.

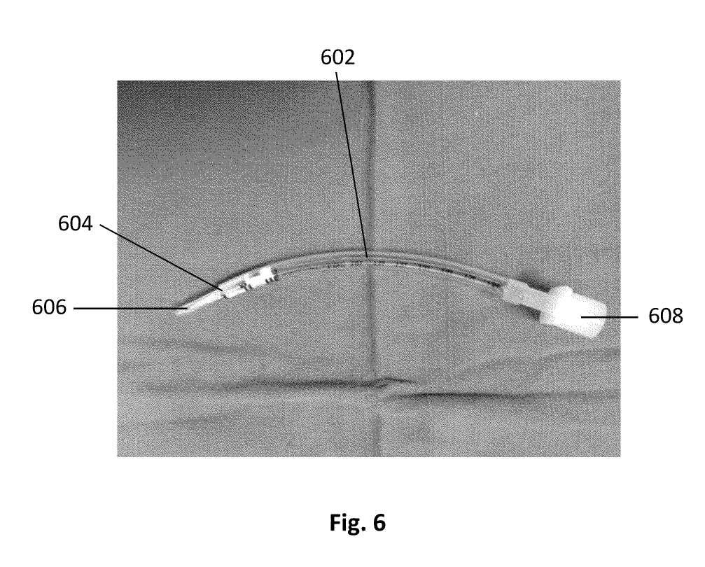

The ET-EB device of Example 1 (illustrated in FIG. 4) is pictured in FIG. 6. The tracheal part of the device 602 branches to the bronchial parts 606 at the distal end of the device via the transition/bifurcation 604. The device is not optimally low-profile, in that the bifurcation transition is not optimally smooth or gradual. A universal connector 608 is seen at the proximal end of the device.

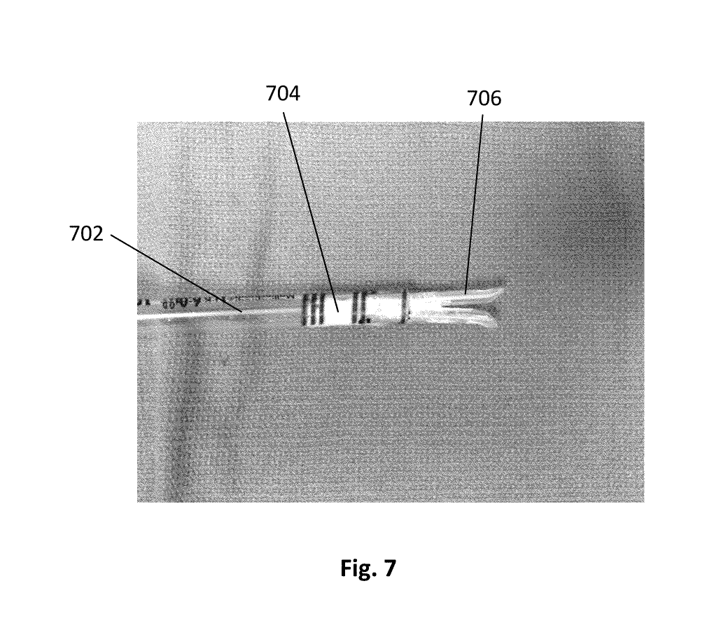

A close-up of the distal end of the first ET-EB device shown in FIG. 6 (and described in Example 1) is shown in FIG. 7. The tracheal tube part 702 transitions at the junction 704 to the bronchial tube parts 706. It can be seen that the junction between the tracheal part and the bronchial parts is a gradual transition and although not optimal still a relatively low-profile device. The opaque adhesive is seen at the transition 704.

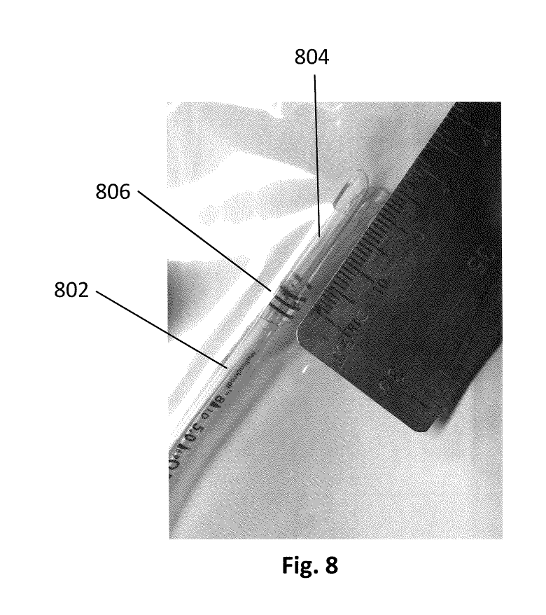

One embodiment of a tooled low-profile ET-EB tube is shown in FIG. 8. A device very similar to this embodiment was used in the third iteration of the extensive patient treatment detailed in Example 1. In this device, the tracheal tube 802 is constructed from the standard 5.0 ETT with two 3.0 endobronchial tubes 804. Note the smoother transition zone 806.

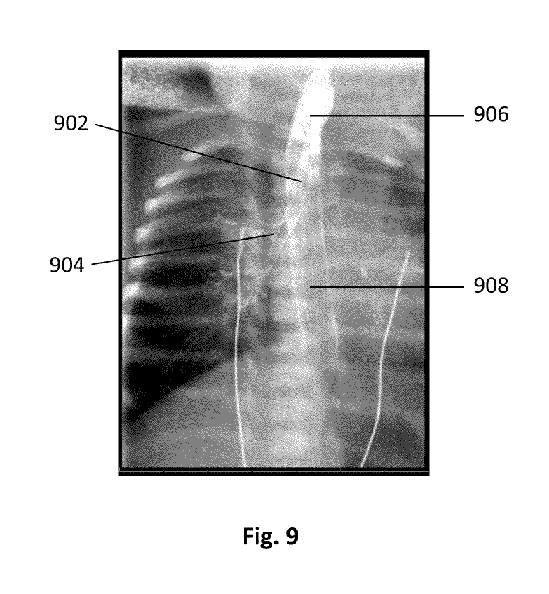

A barium esophagogram of the patient described in Example 1 is shown in FIG. 9. This image shows reflux (and aspiration into the airways) of barium delivered to the stomach via replogle tube. The barium is seen in the trachea 902 and bronchial tree 904 as well as the upper esophagus 906 and lower esophagus 908.

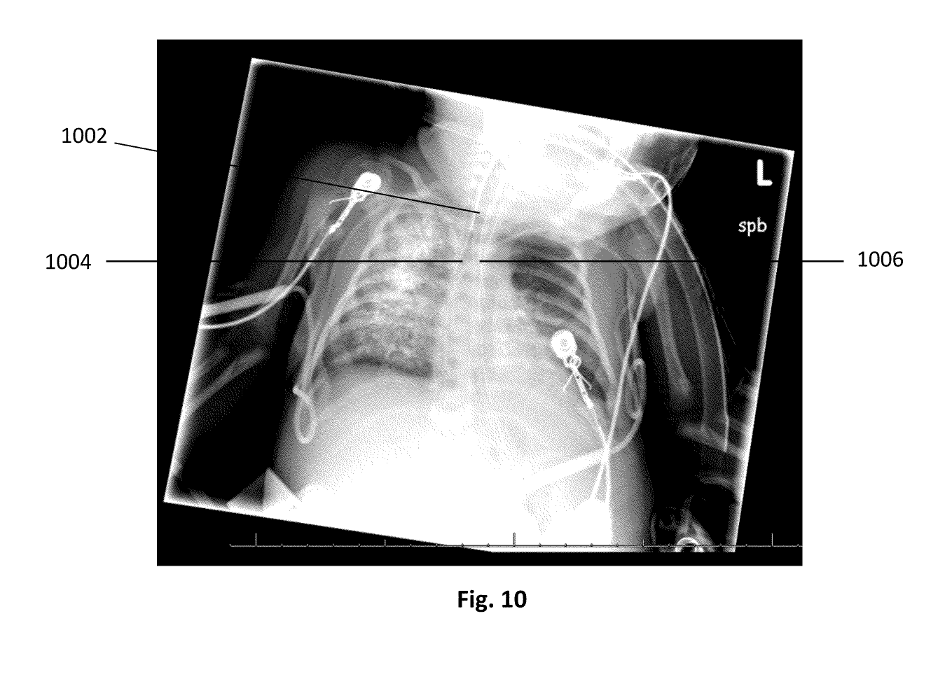

The second version (of three iterations) of an ET-EB tube 1002 is visible in the chest X-ray of the patient described in Example 1, as shown in FIG. 10. The tracheal tube 1002 bifurcates at the patient's carina into the right 1004 and left 1006 endobronchial tubes within the mainstem bronchi. The radio-opaque markings improve visibility.

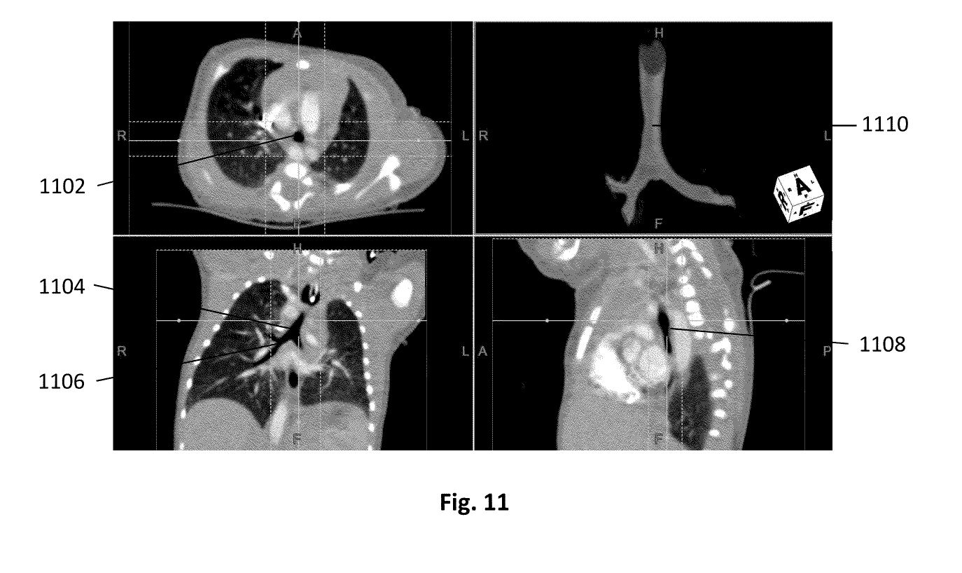

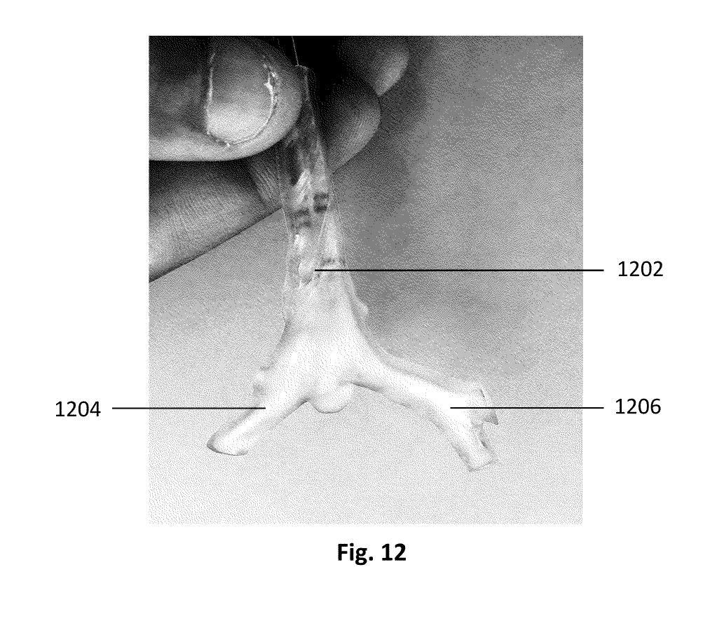

Images of neonatal airways showing tracheo-esophageal fistula (TEF) were reconstructed using 3D computed tomography (CT) and are shown in FIG. 11. The upper left panel is a transverse image, the lower left panel is a coronal image, and the lower right panel is a sagittal image. The trachea is visible in each panel 1102, 1104, 1108 and 1110. The right primary bronchus 1106 is also visible in the lower left panel. These images were used to generate models, such as the airway in the upper right panel which was used to 3D print the model shown in FIG. 12, for placement testing with a prototype representing one embodiment of the ET-EB tube of the invention, as described in Example 4 herein.

A model neonatal TEF airway, shown in FIG. 12, was 3D-printed from the upper right panel 1110 of FIG. 11, for use in a model for placement testing of the prototype ET-EB tube, as further described in Example 4. The printed model includes a neonatal-sized trachea 1202 and its bifurcation to neonatal-sized right 1204 and left 1206 primary bronchi. This 3D-printed airway was used as the lower airway portion of the model, in conjunction with a LAERDAL® Neonatal Intubation Mannequin, which represented the upper airway portion of the model. Experienced anesthesiologists used the model to test placement of the prototype ET-EB tube as compared to a standard endotracheal tube (ETT) of the same size.

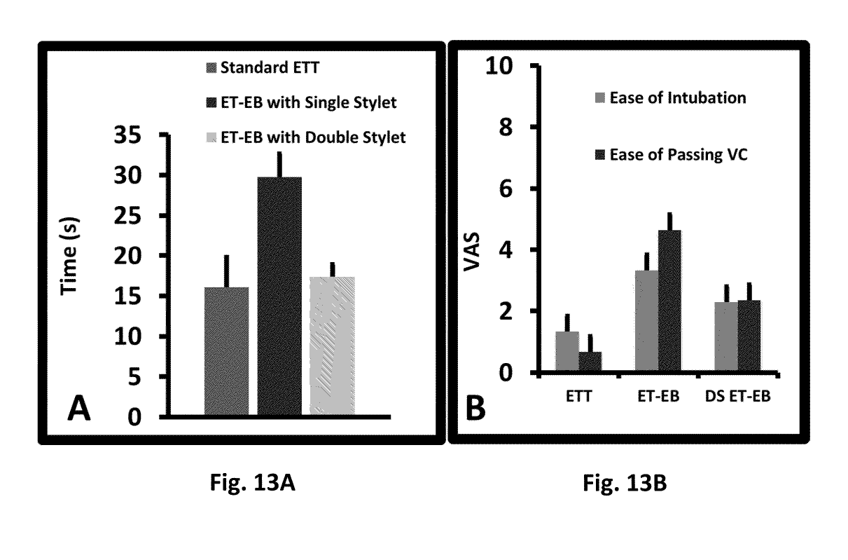

Placement testing for the prototype ET-EB tube and standard ETT (i.e., having only a single tracheal lumen) for comparison was conducted with a single stylet and a double stylet. As shown in FIG. 13A, using a single stylet, the time to placement of the prototype ET-EB tube was longer than a standard ETT (p<0.011). When a double stylet was used to facilitate placement, however, there was no difference in time to placement (p=0.69). The anesthesiologists also subjectively rated difficulty of placement for the tubes using a visual analog scale (VAS). These ratings included separate consideration of the difficulty involved in intubation and in passing the vocal fold (VC). As shown in FIG. 13B, when a single stylet was used to facilitate placement, the physicians perceived that the prototype ET-EB tube was more difficult to place than the standard ETT having only a single tracheal lumen. When a double stylet was used, however, the perceived difficulty was no greater for the prototype ET-EB tube than for the standard ETT, indicating that the ET-EB tube of the invention is suitable for medical procedures that typically require ET and/or EB intubation.

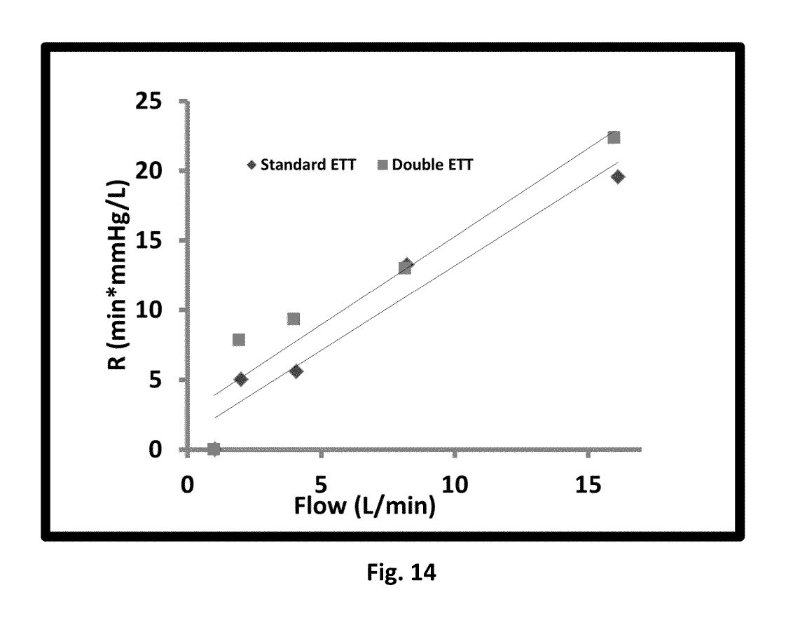

The static pressure relationships of flow and resistance were measured for the prototype ET-EB tube as compared to the standard ETT. Although resistance measured slightly higher for the prototype ET-EB tube, as shown in FIG. 14, the difference was not clinically significant. As a result, no special adjustments to the ventilator should be needed when the ET-EB tube, as compared to an ETT, is used.

### EXAMPLES

### Example 1

### Airway Management for Newborn with Type IV LTEC

Laryngotracheoesophageal cleft (LTEC) results from arrested development of the tracheoesophageal septum and failed posterior fusion of the cricoid lamina during embryogenesis during weeks 5-6 of gestation and generating a spectrum of disease. LTEC has been fatal to many newborns. There are variations of LTEC, and it has been subject to multiple classification schemes, the most common and unifying proposed by Benjamin and Inglis in 1989. Grade IV LTEC extends distally into the thorax, to or even beyond the carina. Presentation is primarily through respiratory symptoms, including aspiration, recurrent pneumonia, stridor, and cyanosis; onset and severity generally correlate with the extent of the cleft. Other developmental abnormalities or syndromes are frequent.

Type IV Laryngotracheoesophageal cleft (LTEC) is notoriously associated with significant morbidity and mortality. Repair presents unique challenges to the anesthetic, surgical and critical care teams. The common lumen shared by the esophagus and trachea confers instability to traditional techniques for maintenance of the airway peri-operatively. Strategies employed to overcome these challenges include standard intubation of the common lumen, tracheostomy, unilateral mainstem bronchial intubation with single lung ventilation, bilateral mainstem bronchial intubation with endotracheal tubes, custom-made bifurcated ETT, laryngeal mask airway, ECMO, and cardiopulmonary bypass.

This example describes the initial use and dynamic redesigning of a low-profile, endotracheal-endobronchial tube for airway management in a patient with extensive Type IV LTEC.

A full-term newborn female was transferred to the Neonatal Intensive Care Unit on the first day of life for evaluation for esophageal atresia (EA) and tracheoesophageal fistula (TEF). Followed by the Maternal-Fetal Medicine and Pediatric General Surgery teams prenatally for polyhydramnios, ultrasound and prenatal fetal MRI revealed dilated segments of the proximal, mid, and distal esophagus with small decompressed stomach, which favored the prenatal diagnosis of EA with TEF. The patient was delivered via cesarean section after 38 weeks of gestation with Apgar scores at 1 and 5 minutes of 6 and 7. A replogle tube passed easily into the stomach and was visualized in the left upper quadrant; air insufflation resulted in distal bowel gas pattern on radiograph, decreasing suspicion of EA.

On the fifth day of life, the patient underwent a barium esophagogram, in which only 5 mL of barium contrast was introduced through the replogle but resulted in immediate reflux with aspiration that required intubation for acute respiratory failure (See FIG. 9). Presence of an H-type TEF was then assumed and repair was planned for the following day.

At the start of the surgical case, a large air leak was noted from the 3.0 cuffless endotracheal tube (ETT). Diagnostic direct laryngoscopy and rigid bronchoscopy using a 4 mm 0-degree HOPKINS® telescope and the Storz AIDA® system revealed LTEC extending to the carina; the mainstem bronchi were widely patent but no membranous trachea was present. Rigid esophagoscopy revealed that the distal esophagus was anatomically normal. Externally, the light of the HOPKINS® telescope at the carina suggested that the apex of the LTEC was near the level of the sternal notch or just behind the manubrium. The planned repair was aborted.

On day 12 of life, the patient returned to the operating room (OR) for planned primary LTEC repair with antecedent Nissen fundoplication and placement of a gastrojejunostomy tube for feeding. The airway was secured with a uniquely fashioned bifurcated endotracheal-endobronchial tube (ET-EB tube). The initial custom ET-EB Tube was constructed from a 6.0 MALLINCKRODT® ETT and two 2.5 MALLINCKRODT® ETTs. The 6.0 ETT was trimmed with a 10-blade scalpel at the single hash-mark, saving the proximal tube. The 2.5 ETTs were trimmed across the double hash-mark, saving the distal tip. The two 2.5 ETTs were then placed inside the 6.0 ETT, advancing them until their single and double hash-marks aligned with the hash-marks on the 6.0 ETT. (See FIGS. 4 and 6) This was then treated with KRAZY GLUE® BRUSH ON® in an off-label use. The BRUSH ON® formula was chosen to eliminate the spill onto the fingers during application. To facilitate curing (or drying) of the KRAZY GLUE® BRUSH ON® into a solid state, the entire unit was submerged in isopropyl alcohol. As the curing process began, a visual change was noted in the KRAZY GLUE® solution from clear to cloudy, and the entire unit was then rinsed in sterile water. Once curing was completed within a few minutes, the 2.5 ETT tips were checked for stability and the tube was then maintained clean until use.

The original device was placed under rigid bronchoscopic guidance with the HOPKINS® rod, and endobronchial intubation was achieved with the use of stylet direction for each lumen. End-tidal carbon dioxide and bilateral breath sounds were present immediately. This intubation yielded saturation rates of 100% throughout the central line and abdominal procedures.

The type IV LTEC was then repaired using an anterior approach via laryngofissure and tracheotomy with right clavicular periosteum harvested as an interposition graft. During this stage of the repair, the oral ETT tube was removed in favor of the use of a wire-reinforced anterior direct bronchial intubation for single-lung ventilation. At the conclusion of the esopheageal and posterior tracheal repairs, a 3.0 cuffed naso-ETT was placed, over which the repair was closed anteriorly. (A smaller ET-EB tube could not be fashioned with existing materials.) A chest x-ray was obtained to optimize positioning of the tube, which was then secured, and the patient was transferred to the PICU.

The post-operative course was complicated by diminishing breath sounds, naso-ETT tube manipulation, inflation of the cuff to seal leaking, and bilious reflux with bile present in the naso-ETT. Planned direct laryngoscopy and bronchoscopy on post-operative day 7 revealed complete dehiscence of the repair. The patient was reintubated orally with the cuffed 3.0 ETT and returned to the PICU. Management of the airway was complicated by air leak. Therefore, at two weeks post-operatively when she returned to the operating room for definitive management of the severe gastroesophageal dysmotility marked by complete atony of the esophagus, stomach and proximal duodenum, she was reintubated with a new hand-made ET-EB tube. At this exploratory laparotomy, extensive adhesive disease, a fistulous communication between a proximal jejunal loop and the duodenum, and an internal hernia were identified. Though the Nissen fundoplication was intact, it was lengthened to provide better control of reflux. The gastrostomy was revised, necrosing small bowel resected, appendix removed, a Roux-en-Y loop created with separate feeding jejunostomy placed, and a second Broviac central venous line was placed. The patient returned to the PICU with the custom ET-EB tube for airway control and protection. She was managed with the ET-EB tube exclusively until she could return to the operating room for re-repair of the LTEC at a 7 week interval after the primary repair. The tube was replaced once after 5 days when it migrated proximally; it was again replaced after another migration proximally a week later, modified for larger endobronchial tubes (6.5->3.0).

The patient remained on ventilatory assistance and intubated with the modified ET-EB Tube uneventfully over the next month. The endobronchial tubes were easily followed on chest X-ray, as shown in FIG. 10. She underwent re-repair of the type IV LTEC employing elective extracorporeal membrane oxygenation (ECMO). Venovenous flow was inadequate due to small vessels, so she was placed on veno-arterial ECMO. Re-repair of the type IV cleft ensued via a trans-cervical approach with cricotracheal separation, thymectomy to improve visualization, and stenting intubation with a standard cuffless 3.5 ETT.

ECMO was complicated by progressive coagulopathy. She was taken to the OR on post-operative day 5 to remove secretions and clot from the right lung, before direct intubation and jet ventilation. On post-operative day 7, tracheal positioning of the 3.5 cuffless ETT with jet ventilation resulted within hours in superior migration of the ETT and a marked decrease in the effectiveness of ventilation efforts in the left lung. Addressing concerns of inadequate oxygenation and ventilation, the patient was re-intubated the next day with a custom-tooled ET-EB Tube of the invention, modified for smoother internal and external tube transitions and a smaller tracheal segment with comparatively larger bronchial segments (4.0->3.0). This tube was used with the jet ventilator. The patient was stable for discontinuation of ECMO four days later, when jet ventilation was discontinued and the patient was transitioned to pressure control ventilation. The patient was extubatable by 19 days post-re-repair with good bilateral breath sounds.

Lung damage secondary to repeated aspiration of gastric and pharyngeal substances is a major concern for patients with tracheo-esophageal defects, and aggressive strategies to protect the lungs are indicated for these patients. Long term protection of the lungs from aspiration-induced damage is best accomplished with repair of the defect, which challenges airway maintenance and oxygenation pre-, intra-, and post-operatively. The stability and the oxygenation and ventilation performance of the ET-EB Tube, along with its ease of construction using commonly available materials, make this a safe and effective method for airway maintenance in patients with extensive tracheo-esophageal defects.

Development and use of the “ET-EB Tube” for this patient granted a stable airway with good oxygen saturations and excellent ventilation acutely in the operative setting and chronically in the intensive care unit.

### Example 2

### Construction of “Tooled” Device of Example 1

Two small tubes (size 3.0), cut near the distal end, were loaded on a mandrel, side-by-side, and wedged inside the proximal end of a larger tube (size 4.0), also cut near the distal end. The mandrel was heated sufficiently to fuse the three tubes together by melting and thereby formed a fused airtight tube junction with the bigger tube. No additional adhesive was needed to secure and seal the transition/junction, and in addition to being a lower-profile tube, the device is advantageously transparent, as compared to the hand-made predecessor versions, in which the adhesive became opaque upon drying and thus obstructed the view of some structures.

### Example 3

### Managing Difficult Airway in Cardio-Thoracic Procedures

In another example, a low-profile ET-EB tube may be used to manage the airway of a patient afflicted with a traumatic injury, surgical, cancerous, infectious, or other condition that compromises the proper functioning of the trachea and/or bronchi.

An adult trauma patient goes to the operating room following a car accident for emergency treatment of a traumatic tear of the membranous trachea extending into the bilateral mainstem bronchi. Traditionally, this is repaired using alternating selective mainstem intubations (complicated in the trauma patient by the rapidly progressive hypercarbic acidosis of hypoventilation that develops with single lung ventilation) or by placement of the patient onto cardiopulmonary bypass (frequently contraindicated in such an injury as it would be a marker of significant other hemorrhagic injury). An adult-sized ET-EB tube with cuffs and valves is placed for control of the trachea-bronchial tree at induction of general endotracheal anesthesia and then throughout repair, obviating the need for either traditional technique for oxygenation and ventilation. The ET-EB tube allows the medical team to avoid the risks of cardiopulmonary bypass (hemorrhage, stroke, toxicity of the system), and it also facilitates the transitions from right to left lung selective ventilation without changing the tube (as needed with existing devices) and provides for rapid control of the lung selection by the anesthesiologist or surgeon.

### Example 4

### Placement Trials of ET-EB Prototype Tube

Tracheo-Esophageal Fistulas (TEFs) and Laryngo-Tracheo-Esophageal Clefts (LTECs) are somewhat rare but occur in live births and must be addressed immediately. These conditions occur as a result of failed separation of segments of the airway from the upper digestive tract or of failure of fusion of the posterior aspect of the airway. Airway management during surgeries for these patients can be challenging. As described above, a bifurcated endotracheal-endobronchial (ET-EB) tube has been used twice to direct air into bronchi, selectively avoiding gastric ventilation and the resulting reflux/aspiration in patients undergoing LTEC repair. The ET-EB prototype tube tested in this study has a lower profile than prior tubes prepared for such cases and allows specific endobronchial ventilation as opposed to endotracheal ventilation (See e.g., FIG. 1).

This study tested difficulty of placement for the novel low-profile bifurcated ET-EB prototype as compared to a standard endotracheal tube (ETT). The study also addressed whether correction would be needed for the use of the new ET-EB prototype with mechanical ventilation. An intubating stylet is a malleable metal wire designed to be inserted into the endotracheal tube to make the tube conform better to the upper airway anatomy of the specific individual. A stylet is commonly used with a difficult laryngoscopy. This study involved placement trials using either a single stylet or a double stylet.

Eight pediatric fellowship-trained anesthesiologists, each having more than 2 years of clinical practice experience, conducted placement trials with the low-profile bifurcated ET-EB prototype tube and a standard uncuffed ETT (i.e., having only a tracheal lumen) of the same size (Mallinckrodt, USA). Simulated patients were modeled with a combination of mannequins and 3D-printed airways. The upper airway was modeled with a LAERDAL® Neonatal Intubation Mannequin (Laerdal Medical, USA). The lower airway was modeled with 3D-printed reconstructions of neonatal TEF airways from larynx to first order bronchi [Software: AquariusNET version 4.4.11 (TeraRecon, Inc., San Mateo, Calif.) and Mimics version 17 (Materialise, Leuven, Belgium); Hardware: Formlabs Desktop Stereolithography 3D printer] See FIGS. 11-12.

Each physician attempted 10 blind placements. Measurements obtained included number of successful attempts, time for placement through the vocal cords (VC) using a Miller #1 laryngoscope and single or double stylet, and subjective assessment of overall ease of placement and ease of passing the VC using a 10-point visual analog scale (VAS). Mechanical flow properties were also measured and reported as mean±standard deviation (SD). ANOVA was used with corrections for multiple comparisons.

With the single stylet, the time to placement of the ET-EB was longer than the standard ETT (p<0.011). However, when a double stylet (DS) was used in the ET-EB, there was no difference in time to placement (p=0.69) (FIG. 13A). Ease of placement and passing the vocal fold was rated more difficult with the ET-EB with the single stylet (p<0.05) but not with the double stylet (FIG. 13B) Average passes/attempts to achieve correct placement with both lumina in the mainstem bronchi was 1.6. The overall success rate was 79%. These results indicate that the ET-EB tube of the invention is suitable for use in medical procedures where ET-EB intubation is indicated.

The static pressure relationships of flow and resistance are shown in FIG. 14. While the resistance was slightly higher in the ET-EB compared to the standard ETT, this difference was not clinically significant.

The ET-EB tube showed no difference in time or overall ease of placement when using double stylets. Two stylets also allowed for control of tips through vocal cords and into bronchi. The observed 79% successful distal placement rate can be improved with endoscopic airway assessment for in vivo studies.

Regarding pressure adjustments, it appears that there are no ventilator adjustments or corrections needed for the use of the ET-EB clinically, again indicating that the ET-EB tube of the invention is suitable for use in medical procedures where ET-EB intubation is indicated. These trials may be supplemented with in vivo studies for objective and subjective measurement of perioperative outcomes.

Again, as will be appreciated, the foregoing examples are illustrative, and non-limiting, of the many possible embodiments of the present invention.

1. A device for managing a patient airway, comprising:
a single-lumen tracheal tube that bifurcates to two single-lumen bronchial tubes, and
a substantially smooth exterior surface.

2. The device of claim 1, further comprising a substantially smooth interior surface.

3. The device of claim 1, wherein the device is selected or customized for the anatomy of a particular patient.

4. The device of claim 1, further comprising an opening to accommodate the level of a right superior lobar bronchus as it branches from the right main bronchus.

5. The device of claim 4, wherein the opening is on the bifurcated part of the device.

6. The device of claim 1, further comprising an external cuff.

7. The device of claim 1, further comprising an internal valve.

8. The device of claim 7, wherein the internal valve comprises an internal cuff.

9. The device of claim 1, wherein the internal diameter of the tracheal tube part of the device is between 2.0-8.5 mm.

10. The device of claim 1, wherein the device is substantially transparent.

11. The device of claim 9, wherein the size and/or ratio between tracheal and bronchial diameters are designed to fit within neonatal, early childhood, pediatric, adolescent, adult, or large or enlarged adult size trachea and bronchi.

12. The device of claim 1, wherein the device is substantially airtight at the junction where the tracheal tube bifurcates to two bronchial tubes.

13. The device of claim 1, wherein the bronchial tubes are asymmetrical.

14. A method for managing an airway, comprising placement of a device having a single-lumen tube that bifurcates to two single-lumen tubes and having substantially smooth exterior surfaces.

15. The method of claim 14, further comprising selecting or customizing the device to comprise an opening corresponding to a superior lobar bronchus of the patient.

16. The method of claim 14, further comprising using an inflatable cuff positioned on a bronchial tube to secure the device in position once it is placed.

17. The method of claim 14, further comprising using an internal valve to limit gas flow in a particular segment of the device.

18. The method of claim 14, wherein the method is used to manage the airway of a patient afflicted with a congenital defect, traumatic injury, surgical, cancerous, infectious, or other condition that compromises the proper functioning of the trachea and/or bronchi and/or to manage the airway of a patient before, during, or after a cardio-thoracic procedure.

19. The method of claim 14, wherein gases are delivered selectively to one lung.

20. The method of claim 14, further comprising recruitment and/or recovery of a lung during or following the use of ECMO or cardiopulmonary bypass, or to facilitate avoidance of such procedures.
